(19) United States
(12) Patent Application Publication
(10) Pub. No.: US 2024/0123097 A1
(43) Pub. Date: Apr. 18, 2024

(54) Dual-Mode Pharmaceutical Composition for Peptide-Specific Targeted Positron Emission Tomography and Boron Neutron Capture Therapy and the Use Thereof

(71) Applicant: Primo Biotechnology Co., Ltd, Taipei City (TW)

(72) Inventor: Ya-Yao Huang, Taipei City (TW)

(73) Assignee: Primo Biotechnology Co., Ltd, Taipei City, TW (TW)

(21) Appl. No.: 18/473,324

(22) Filed: Sep. 25, 2023

Related U.S. Application Data
(60) Provisional application No. 63/377,051, filed on Sep. 26, 2022.

(51) Int. Cl. A61K 51/08; A61K 38/17; A61K 38/48; C07K 14/47; C07K 14/705; C12N 9/48

(52) U.S. Cl. CPC A61K 51/08; A61K 38/1709; A61K 38/177; A61K 38/4813; C07K 14/4725; C07K 14/705; C12N 9/485; C12Y 304/14005

(57) ABSTRACT

A pharmaceutical composition for Positron Emission Tomography (PET) and Boron Neutron Capture Therapy (BNCT) and the use for treating cancer thereof are provided, wherein the pharmaceutical composition comprises a compound having structure of formula I, wherein R1 is a specific peptide, one of R2, R3, and R4 is Fluorine-18 (¹⁸F) or Fluorine-19 (¹⁹F), one of the other two position is —B(OH)₂, and the remaining position is hydrogen (H); a method for preparing a compound having structure of formula I is also provided in the present invention.

### TITLE OF INVENTION

Dual-Mode Pharmaceutical Composition for Peptide-Specific Targeted Positron Emission Tomography and Boron Neutron Capture Therapy and The Use Thereof

### FIELD OF THE INVENTION

The present invention is related to a pharmaceutical composition for peptide-specific targeted positron emission tomography and boron neutron capture therapy and the use thereof. Particularly, a pharmaceutical composition for peptide-specific targeted positron emission tomography and boron neutron capture therapy including specifically targeted peptides such as inhibitor derivatives of prostate-specific membrane antigen (PSMA), fibroblast activation protein (FAP) or heparan sulfate proteoglycans (HSPGs), etc.

### BACKGROUND OF THE INVENTION

Boron Neutron Capture Therapy (BNCT) is a targeted radiation therapy in which a patient is given a boron-containing drug, such as Boronophenylalanine (BPA), which uses the characteristics of boron to absorb neutrons and produce nuclear reactions, and then divides into lithium and alpha particles, just like burying and detonating a nuclear bomb inside a tumor to achieve the effect of destroying cancer cells, but its damage to surrounding tissues is extremely low.

BNCT is extremely lethal to cells, so it is necessary to precisely position the location of cancer. Therefore, BNCT is usually preceded by Positron Emission Tomography (PET) technology. The so-called PET is to first administer boron-containing drugs containing radioactive isotopes such as gallium, fluorine, and carbon (e.g., ¹⁸F-BPA) to an individual for whole-body or specific tumor observation to screen out the site that need to be treated, and then BNCT is carried out to ensure that boron-containing drugs can accurately reach the lesion, and then boron neutron capture treatment is performed.

However, there are still molecular structural differences between BPA and ¹⁸F-BPA, so it still needs to be confirmed whether their pharmacological efficacy are the same.

### SUMMARY OF THE INVENTION

The present invention is related to a pharmaceutical composition for Positron Emission Tomography (PET) and Boron Neutron Capture Therapy (BNCT), which comprises a compound having structure of formula I:

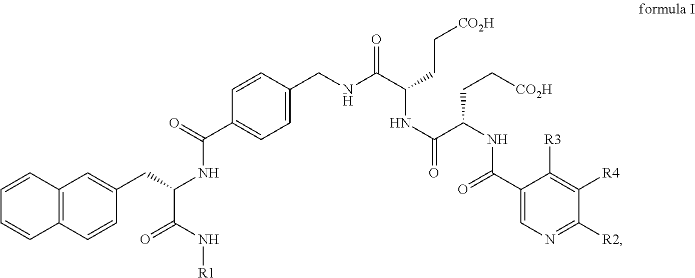

and pharmaceutically accepted carriers thereof; wherein R1 is a specific peptide, one of R2, R3, and R4 is Fluorine-18 (¹⁸F) or Fluorine-19 (¹⁹F), one of the other two position is —B(OH)₂, and the remaining position is hydrogen (H).

The present invention is also related to a method for preparing a compound having structure of formula I, comprising:
a. providing a compound having structure of formula II:

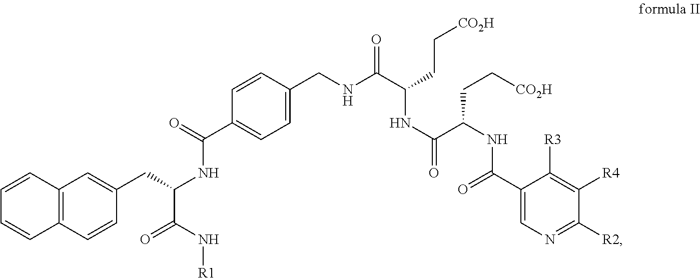

wherein, R1 is a specific peptide, one of R2, R3, and R4 is Fluorine-18 (¹⁸F) or Fluorine-19 (¹⁹F), one of the other two position is a halogen atom other than fluorine or pinacol boronate derivative, such as diazaborinane derivative, BXₙ, or BX−M+(X represents any functional group, such as halogen. n is 2 to 4. and M+represents monovalent monoatomic cation, a 3-to 10-membered ring, or a 3-to 10-membered ring polyatomic cation, or complex cation), and the remaining position is hydrogen (H);
b. treating with pinacol boronate reagent; and
c. obtaining the compound having structure of formula I:

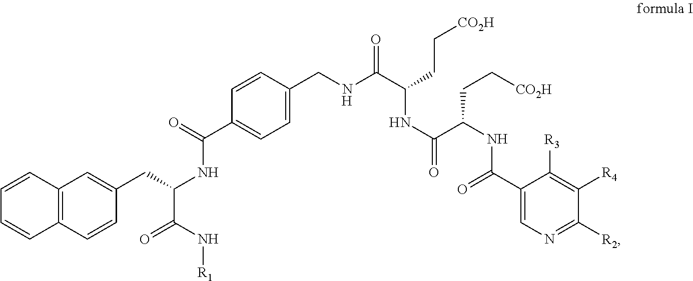

wherein R1 is a specific peptide, one of R2, R3, and R4 is Fluorine-18 (¹⁸F) or Fluorine-19 (¹⁹F), one of the other two position is —B(OH)₂, and the remaining position is hydrogen (H).

The present invention is also related to a use of a compound in the preparation of a pharmaceutical composition for treating cancer, wherein the composition comprises the compound having structure of formula I:

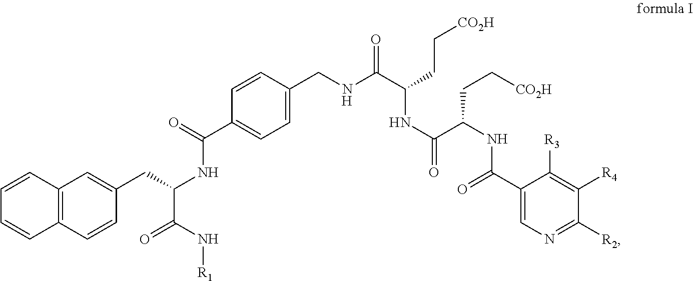

wherein R1 is a specific peptide, one of R2, R3, and R4 is Fluorine-18 (¹⁸F) or Fluorine-19 (¹⁹F), one of the other two position is —B(OH)2, and the remaining position is hydrogen (H).

### DETAILED DESCRIPTION OF THE INVENTION

In order to solve the pharmacological efficacy doubts arising from the difference in molecular structure between BPA and ¹⁸F-BPA and combine with cancer-specific therapeutic purposes, the applicant of the present invention investigated a new pharmaceutical composition for Positron Emission Tomography (PET) and Boron Neutron Capture Therapy (BNCT).

The present invention is related to a pharmaceutical composition for Positron Emission Tomography (PET) and Boron Neutron Capture Therapy (BNCT), which comprises a compound having structure of formula I:

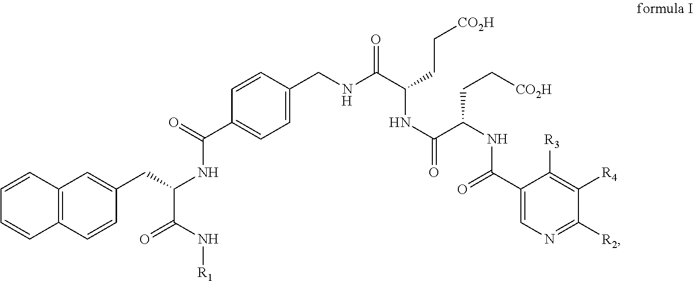

and pharmaceutically accepted carriers thereof; wherein R1 is a specific peptide, one of R2, R3, and R4 is Fluorine-18 (¹⁸F) or Fluorine-19 (¹⁹F), one of the other two position is —B(OH)2, and the remaining position is hydrogen (H).

Preferably, the pharmaceutical composition for PET and BNCT of the present invention is used for PET or BNCT.

Most preferred, the pharmaceutical composition for PET and BNCT of the present invention can be used for PET or BNCT simultaneously.

The present invention is also related to a method for preparing a compound having structure of formula I, comprising:
a. providing a compound having structure of formula II:

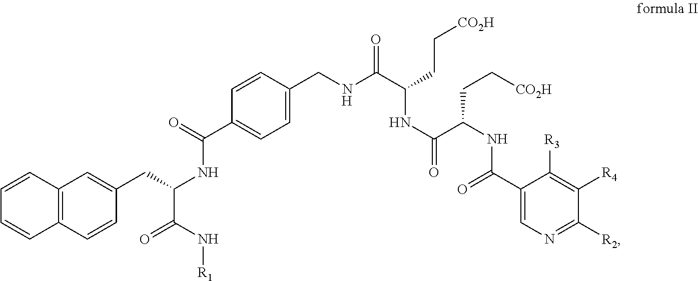

wherein, R1 is a specific peptide, one of R2, R3, and R4 is Fluorine-18 (¹⁸F) or Fluorine-19 (¹⁹F), one of the other two position is a halogen atom other than fluorine or pinacol boronate derivative, such as diazaborinane derivative, BXₙ, or BX−M+(X represents any functional group, such as halogen. n is 2 to 4. and M+represents monovalent monoatomic cation, a 3-to 10-membered ring, or a 3-to 10-membered ring polyatomic cation, or complex cation), and the remaining position is hydrogen (H);
b. treating with pinacol boronate reagent; and
c. obtaining the compound having structure of formula I:

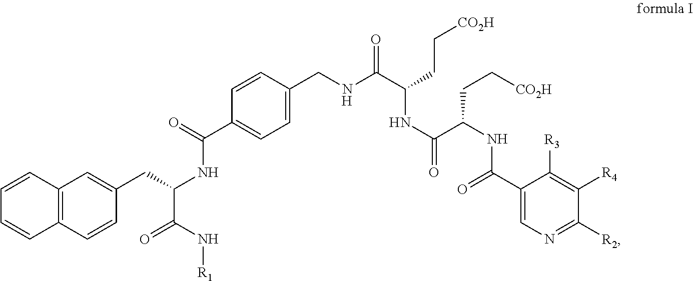

wherein R1 is a specific peptide, one of R2, R3, and R4 is Fluorine-18 (¹⁸F) or Fluorine-19 (¹⁹F), one of the other two position is —B(OH)2, and the remaining position is hydrogen (H).

In addition, the present invention is also related to a use of a compound in the preparation of a pharmaceutical composition for treating cancer, wherein the composition comprises a compound having structure of formula I:

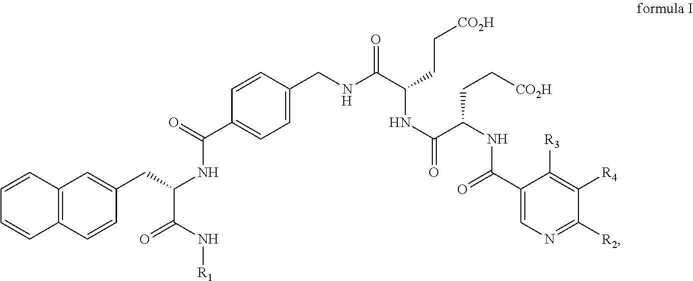

wherein R1 is a specific peptide, one of R2, R3, and R4 is Fluorine-18 (¹⁸F) or Fluorine-19 (¹⁹F), one of the other two position is —B(OH)₂, and the remaining position is hydrogen (H).

Preferably, in the present invention, the specific peptide is inhibitor derivatives of prostate-specific membrane antigen (PSMA), fibroblast activation protein (FAP) or heparan sulfate proteoglycans (HSPGs).

Most preferred, in the present invention, the specific peptide is prostate-

specific membrane antigen (PSMA).

The present invention is also related to a method for treating cancer, comprising administering a compound having structure of formula I to a subject suffering from cancer:

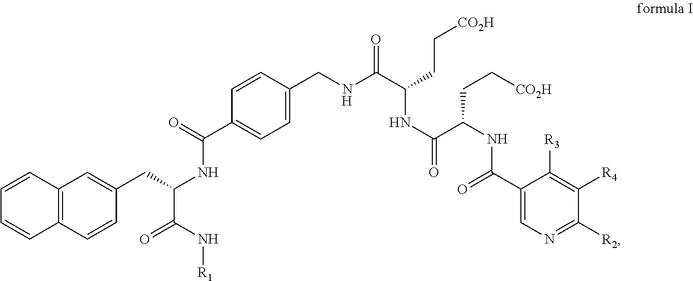

wherein R1 is a specific peptide, one of R2, R3, and R4 is Fluorine-18 (' 8 F) or Fluorine-19 (¹⁹F), one of the other two position is —B(OH)2, and the remaining position is hydrogen (H).

In the present invention, the cancer comprises liver cancer, lung cancer, breast cancer, colorectal cancer, rectal cancer, head and neck cancer, brain cancer, pancreatic cancer, ovarian cancer and prostate cancer.

### EMBODIMENTS

The embodiments merely describe the best example of the present invention, but are not intended to limit the scope of the present invention.

The present invention is related to a pharmaceutical composition for Positron Emission Tomography (PET) and Boron Neutron Capture Therapy (BNCT), which comprises a compound having structure of formula I:

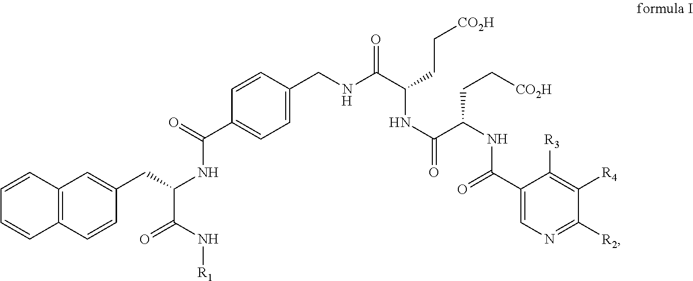

and pharmaceutically accepted carriers thereof; wherein R1 is a specific peptide, one of R2, R3, and R4 is Fluorine-18 (¹⁸F) or Fluorine-19 (¹⁹F), one of the other two position is —B(OH)₂,and the remaining position is hydrogen (H).

In the present invention, for example, if the subject of treatment is prostate cancer, the specific peptide can be an inhibitor derivative of prostate-specific cell membrane-specific antigen (PSMA); if the subject of treatment is colorectal cancer, the specific peptide can be fibroblast activation protein (FAP).

The present invention is also related to a method for preparing a compound having structure of formula I, comprising:
a. providing a compound having structure of formula II:

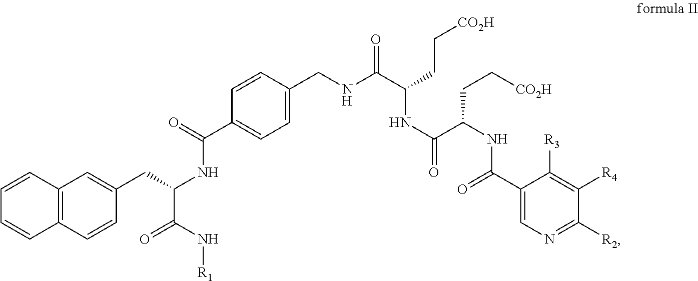

wherein, R1 is a specific peptide, one of R2, R3, and R4 is Fluorine-18 (¹⁸F) or Fluorine-19 (¹⁹F), one of the other two position is a halogen atom other than fluorine or pinacol boronate derivative, such as diazaborinane derivative, BXₙ, or BX−M+(X represents any functional group, such as halogen. n is 2 to 4. and M+represents monovalent monoatomic cation, a 3-to 10-membered ring, or a 3-to 10-membered ring polyatomic cation, or complex cation), and the remaining position is hydrogen (H);
b. treating with pinacol boronate reagent; and
c. obtaining the compound having structure of formula I:

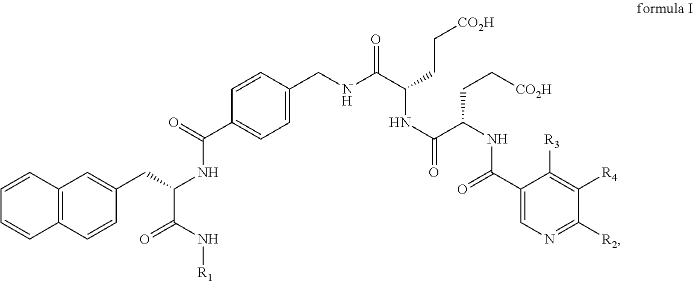

wherein R1 is a specific peptide, one of R2, R3, and R4 is Fluorine-18 (¹⁸F) or Fluorine-19 (¹⁹F), one of the other two position is —B(OH)2, and the remaining position is hydrogen (H).

In another embodiment, the present invention is a use of a compound in the preparation of a pharmaceutical composition for treating cancer, wherein the composition comprises a compound having structure of formula I:

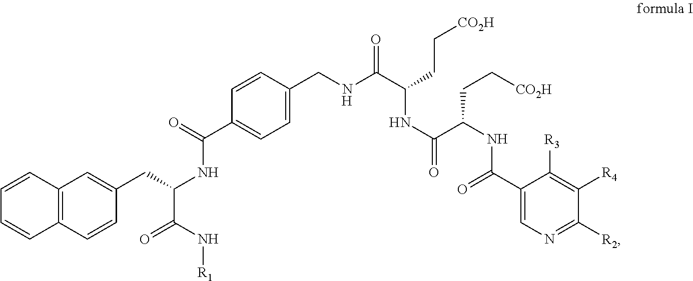

wherein R1 is a specific peptide, one of R2, R3, and R4 is Fluorine-18 (¹⁸F) or Fluorine-19 (¹⁹F), one of the other two position is —B(OH)₂,and the remaining position is hydrogen (H).

In the present invention, for example, if the subject of treatment is prostate cancer, the specific peptide can be an inhibitor derivative of prostate-specific cell membrane-specific antigen (PSMA); if the subject of treatment is colorectal cancer, the specific peptide can be fibroblast activation protein (FAP).

When the pharmaceutical composition of the present invention was used for PET, R2 was fluorine-18 (¹⁸F), in this case, fluorine-18 was marked in the drug by Aromatic Nucleophilic Substitutions. The flow is:

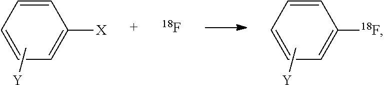

wherein X is a leaving group, such as Halogen, Nitro group (NO₂), Trimethylammonium group (R—N+(CH₃)₃), etc.; Y is an electro-withdrawing group relative to the ortho-position or para-position Group, such as an Acyl group (R—CO), a Cyano group (R—CN), a Nitro group, etc.

After the treatment target was confirmed by PET, in the application of BNCT, fluorine-18 (¹⁸F) of R2 was replaced with fluorine-19 (¹⁹F) through fluorination reaction commonly used in general organic chemistry, so that the molecular structure of compounds in the pharmaceutical compositions used in PET and BNCT were the same, and the pharmacological efficacy were consistent.

Most preferred, in the present invention, the pharmaceutical composition could also be used for PET and BNCT simultaneously.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cells, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method for preparing a compound having structure of formula I, comprising:
a. providing a compound having structure of formula II: 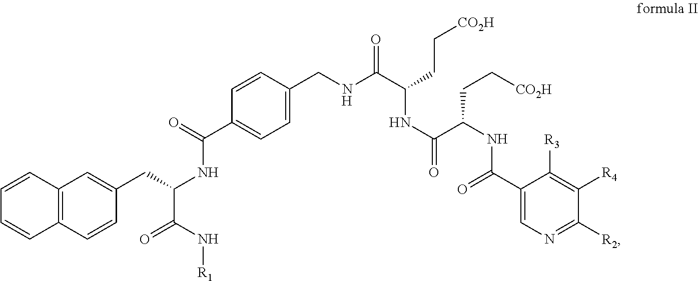
wherein, R1 is a specific peptide, one of R2, R3, and R4 is Fluorine-18 (' 8 F) or Fluorine-19 (¹⁹F), one of the other two position is a halogen atom other than fluorine or pinacol boronate derivative, such as diazaborinane derivative, BXₙ, or BX−M+(X represents any functional group, such as halogen. n is 2 to 4. and M+represents monovalent monoatomic cation, a 3-to 10-membered ring, or a 3-to 10-membered ring polyatomic cation, or complex cation), and the remaining position is hydrogen (H);
b. treating with pinacol boronate reagent; and
c. obtaining the compound having structure of formula I: 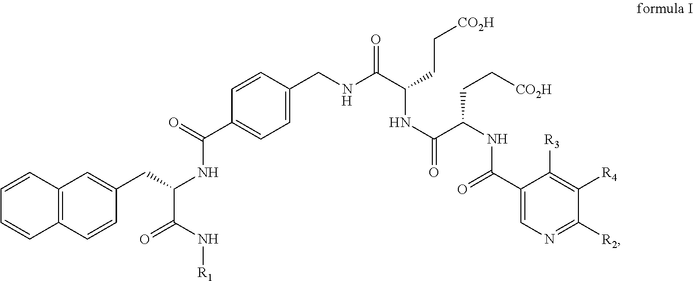
wherein R1 is a specific peptide, one of R2, R3, and R4 is Fluorine-18 (¹⁸F) or Fluorine-19 (¹⁹F), one of the other two position is —B(OH)2, and the remaining position is hydrogen (H).

2. The method of claim 1, wherein the specific peptide is inhibitor derivatives of prostate-specific membrane antigen (PSMA), fibroblast activation protein (FAP) or heparan sulfate proteoglycans (HSPGs).

3. The method of claim 1, wherein the specific peptide is inhibitor derivatives of prostate-specific membrane antigen (PSMA).

4. A pharmaceutical composition for Positron Emission Tomography (PET) and Boron Neutron Capture Therapy (BNCT), which comprises a compound having structure of formula I: 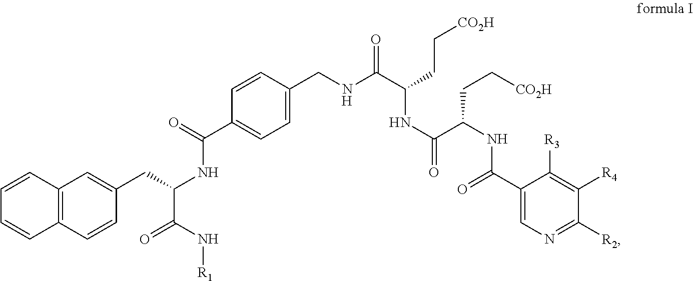
and pharmaceutically accepted carriers thereof; wherein R1 is a specific peptide, one of R2, R3, and R4 is Fluorine-18 (¹⁸F) or Fluorine-19 (¹⁹F), one of the other two position is —B(OH)2, and the remaining position is hydrogen (H).

5. The drug of claim 4, wherein the specific peptide is inhibitor derivatives of prostate-specific membrane antigen (PSMA), fibroblast activation protein (FAP) or heparan sulfate proteoglycans (HSPGs).

6. The drug of claim 4, wherein the specific peptide is inhibitor derivatives of prostate-specific membrane antigen (PSMA).

7. The drug of claim 4, which can be used for PET and BNCT simultaneously.

8. A use of a compound in the preparation of a pharmaceutical composition for treating cancer, wherein the composition comprises a compound having structure of formula I: 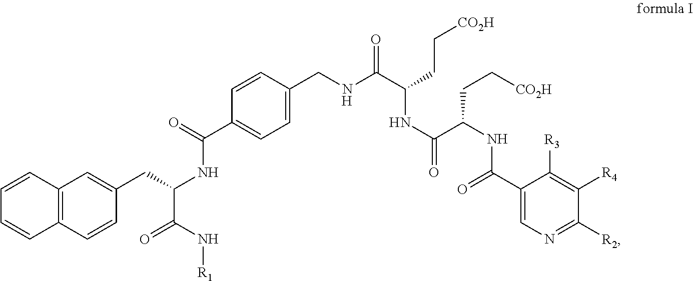
wherein R1 is a specific peptide, one of R2, R3, and R4 is Fluorine-18 (¹⁸F) or Fluorine-19 (¹⁹F), one of the other two position is —B(OH)₂,and the remaining position is hydrogen (H).

9. The use of claim 8, wherein the specific peptide is inhibitor derivatives of prostate-specific membrane antigen (PSMA), fibroblast activation protein (FAP) or heparan sulfate proteoglycans (HSPGs).

10. The use of claim 8, wherein the specific peptide is inhibitor derivatives of prostate-specific membrane antigen (PSMA).

11. The use of claim 8, wherein the cancer comprises liver cancer, lung cancer, breast cancer, colorectal cancer, rectal cancer, head and neck cancer, brain cancer, pancreatic cancer, ovarian cancer and prostate cancer.
